(19) United States
(12) Patent Application Publication
(10) Pub. No.: US 2026/0114908 A1
(43) Pub. Date: Apr. 30, 2026

(54) EXPANDABLE BONE IMPLANT FOR VETERINARY ORTHOPAEDIC SURGERY, ORTHOPAEDIC SYSTEM AND METHOD FOR MANUFACTURING THE IMPLANT

(71) Applicant: IN LIFE VET SA, Bulle (CH)

(72) Inventor: Guillaume LACAZE, La Rippe (CH)

(73) Assignee: IN LIFE VET SA, Bulle (CH)

(21) Appl. No.: 19/340,543

(22) Filed: Sep. 25, 2025

(30) Foreign Application Priority Data
Sep. 25, 2024 (EP) 24202389.3

(51) Int. Cl. A61B 17/70; A61B 17/00; A61F 2/44

(52) U.S. Cl. CPC A61B 17/7097; A61F 2/441; A61B 2017/00526; A61B 2017/00831

(57) ABSTRACT

An expandable bone implant for veterinary orthopaedic surgery for restoring the volume and/or geometry of a bone, to an orthopaedic system and to a method for manufacturing the implant which includes a hollow body formed by a sheet made of a biocompatible metal alloy, closed on itself in a sealed manner, between proximal and distal ends, having, in the folded configuration, a plurality of pairs of folds, each of the pairs comprising an antiform fold, referred to as convex, and possible persistence of a synform fold, referred to as concave, the folds lying on top of each other in the folded configuration so that the surfaces present between each of the convex and concave folds are rolled around the longitudinal axis, the sheet being plastically deformable to allow expansion of the implant from the folded configuration to the deployed configuration, when a fluid is injected into the implant.

The present application relates to the field of surgery, in particular veterinary orthopaedic surgery and in particular to the treatment of a collapsed bone structure by restoring the volume of (or correcting) this bone structure or at least restoring the geometry of a bone structure. The present application relates in particular to an implant and to the method for manufacturing it, as well as to a system for restoring bone structure, in particular in the spine for the treatment (often called “reduction”) of compression fractures, in particular vertebral compression fractures (VCFs).

In this field, the problem of restoring the volume of bone structure that has collapsed is well known and the literature contains an abundance of solutions using expandable implants capable of passing from a folded configuration to an expanded deployed configuration to restore the height of the bone structure, preferably in combination with an injection of bone substitute cement, known as cement (or bone cement). Many cements are known and they all have the advantage of being injectable in a liquid or viscous state for a certain period of time, then of hardening (by polymerization) inside the bone structure in order to stabilize it.

A major problem in this field concerns the expansion of the implant to restore height to the damaged bone tissue. Numerous solutions are known from the prior art, such as in particular from patent applications EP3086729, U.S. Pat. No. 11,540,926, EP3747385, EP2572680, EP3958752, EP2693967, EP2405835, U.S. Pat. No. 9,579,130, EP4216836, WO2023122005, WO2022162418, EP3843668 or U.S. Pat. No. 10,945,861 but these solutions present various problems of difficulty of handling for deploying to the expanded state, and of stability and reliability once deployed. Moreover, these known solutions are generally accompanied by an injection of bone cement, but do not provide any teaching relating to cement leakage outside of the implant, whereas such leakage can be detrimental to the surrounding tissue, or even to the entire organism if the chemical substances in the cement get into the bloodstream. In fact, cement generally comprises one or more polymerizable chemical substances, for example such as poly(methyl methacrylate) (PMMA) and possibly additives. In addition, the temperature reached during the polymerization of the cement is not harmless since it is generally greater than 60°.

Devices for the correction and stabilization (or bone fracture reduction), particularly of the spine in the form of a stent, or in the form of porous inflatable bags or of balloons, as in documents EP1408888 or EP1379185, possibly equipped with support flanges as in document US20060100706, are known from the prior art, in particular from documents EP1308134, U.S. Pat. Nos. 9,510,877, and 8,936,627 or EP2467099. Numerous documents propose this type of stent, that is to say a deformable endoprosthesis similar to vascular stents, extenders or endoprostheses, which are generally in the form of a meshed tubular body, usually metallic and deformable by the introduction of an inflatable balloon to expand the body by spreading the meshes apart, the balloon then being removed to allow the injection of cement, which hardens and thus forms a correcting and stabilizing structure. However, these devices have the drawback of requiring implantation in two stages: the inflation of the balloon and then the injection of cement, which slows down and complicates the operation and also presents a risk of the device collapsing between the deflation of the balloon and filling of the stent with cement. Furthermore, these solutions have the drawback of not addressing the major problem of cement leaks.

Solutions using implants with a mesh structure, made of shape-memory metal, which is constrained into a folded shape for insertion into the bone tissue and capable of expanding spontaneously, when the stress is released and/or under heat, are also known, in particular from documents EP1938765, EP2351539 or WO200434924. These solutions have the drawback of using expensive alloys and complex manufacturing to obtain an adequate shape memory suitable for the intended implantation, which means additional cost when multiplied by the number of different implants necessary to cover the various pathological cases, in particular by the amplitude of the deformation of which the shape memory material is capable. Moreover, the force exerted by the return of the metal to its unstressed form is often not sufficient to properly correct the bone structure which has collapsed, or is at least a limiting factor. Furthermore, these solutions also have the drawback of not addressing the major problem of cement leaks.

Solutions using expandable implants that can be expanded using a lever mechanism, in the manner of a car jack, to restore the bone structure to a determined height are also known from the prior art, in particular from documents EP2405835, U.S. Pat. No. 9,579,130, EP2572680 or EP1956990. These solutions have the advantage of not risking collapse unlike a stent that is deployed by a balloon which is removed before the injection of the cement, but also have the drawback of implantation in two stages and of the fact that the dimensions of the support surface for exerting the expansion force on the bone tissue are limited, in comparison with stents in particular. Furthermore, they also have the drawback of being expensive and of likewise not addressing the major problem of cement leaks.

The problem of cement leakage has already been identified, in particular in documents EP1408888, EP1509175 or WO200394805, which express the advantages that would be offered by a deformable implant that is not very permeable or that is impermeable, so as to limit or prevent cement leakage. These documents envisage numerous solutions for an expandable implant, made of metal or polymer, which could be either soft and flexible such as a membrane or a fabric, or even elastic, or semi-rigid (“conformable”) or rigid, or made of a shape memory material, with a continuous or fenestrated wall (i.e., mesh) and which could be porous or non-porous. However, all these hypotheses described in these documents define, above all, conceivable treatment methods and objectives to be achieved, without providing any real teaching as regards the technical characteristics or the structural arrangement of the implants, or on how to obtain such implants and thus implement these methods. These proposals therefore present a major problem of technical feasibility.

Furthermore, one problem which is not identified in the prior art concerns the cement injection site and the distribution of the forces exerted on the bone tissues to rectify them. Indeed, the impermeability of an implant makes it possible to avoid cement leaks, but the nature of the impermeable membrane and its technical characteristics such as its physico-chemical and mechanical properties influence its ability to deploy without breaking and to rectify the bone structure. Thus, an elastic membrane has the drawback of deforming excessively in low density zones and thus of having a limited capacity to restore height, with, in addition, the risk of breaking at the points at which its maximum elasticity is exceeded because of this uncontrolled deformation. A semi-rigid membrane is therefore preferable, but this problem of deformation also involves a problem of shapes of the implant, in the folded configuration and especially in the deployed configuration. Indeed, the shape of the deployed implant delimits the cement injection site and the control of this site is important for the distribution of the forces leading to the filling of the low density zones while rectifying the structure (especially in terms of height), and this has an impact on the success of the operation. Thus, it will be understood that the provision of an implant that addresses all of these problems is accompanied by a problem regarding technical feasibility and therefore manufacture.

Other recurring problems in orthopaedic surgery include invasiveness (i.e., the goal of making the smallest possible incision and of minimizing lesions) and also the deployment ratio in order to obtain a deployed implant that fills the largest possible volume while having been introduced through the smallest possible passage. Furthermore, this deployment ratio will have an impact on the distribution of forces for rectifying the vertebrae: if the deformability is too great, the cement-injection pressure will deform the pouch rather than restoring height.

A problem complementary to that of deployment concerns folding, which is generally not possible in implants of the prior art. Control of the folding allows control over the deployment and therefore over the injection site with a uniform distribution of the cement and of the pressure to fill the space to be filled following the collapse. Perfect proportionality suited to the fracture while at the same time respecting the shape of the bone inside the fracture is thus achieved.

In this context, it will be understood that there is still in this field a technical problem concerning the restoration of bone structure (rectification or reduction of fracture or increase in volume after collapse) by means of an expandable (deployable) implant that is able to expand collapsed bone tissue and is impermeable enough to prevent or limit the leakage of cement out of the implant with control of the injection site.

Finally, a main problem that still persists in the field concerns the technical feasibility of manufacturing implants proposed in the prior art. Document WO200394805, for example, describes numerous methods of administering substances and in particular bone cement, with numerous variants envisaged for an expandable implant, made of metal or polymer, which could be either soft and flexible such as a membrane or a fabric, or semi-conformable or rigid, or made of a shape memory material, with a continuous or fenestrated (i.e., meshed) wall and which could be porous or non-porous. However, that document describes only conceivable methods of treatment, but does not provide any teaching as to the technical features, or the structural arrangement of these many hypothetical implants used for these envisaged methods, or on how to obtain such implants and thus actually implement these methods. These proposals therefore present a major problem of technical feasibility and define objectives to be achieved rather than means for achieving them. Moreover, although many objectives have been detailed in the literature, many implants proposed for achieving these objectives have never seen the light of day due to problems of manufacture. In order to address the problem of manufacture, it is necessary to take into account the problems related to the desire to compact/fold a “bag” (balloon/pouch) made of a rigid and impermeable material in order to:
pass through a cylindrical duct;
allow the pouch to expand without rupturing, despite the rigidity and the desired difference in volume between the folded volume and the deployed volume;
control the volume and the distribution of expansion forces on the bone.

In this context, one object of the present invention is to overcome at least certain drawbacks of the prior art by proposing an implant for restoring a collapsed bone structure that is reliable and simple to handle and to implant.

This aim is achieved by an Expandable bone implant for veterinary orthopaedic surgery for restoring the volume and/or geometry of a bone by expansion between a folded configuration and a deployed configuration, said implant comprising a hollow body extending along a longitudinal axis between a proximal end connectable to an implantation instrument for holding the implant and a distal end intended to be first inserted into the bone, said implant being characterized in that:
the wall of said hollow body is formed by a sheet made of a biocompatible metal alloy, closed on itself in a sealed manner, between said proximal and distal ends;
said sheet has, at least in the folded configuration, a plurality of pairs of folds, each of the pairs comprising an antiform fold, referred to as convex, and a synform fold, referred to as concave, said folds lying on top of each other in the folded configuration so that the surfaces present between each of said convex and concave folds are rolled around the longitudinal axis;
said proximal end comprises a ring secured, in a sealed manner, to the lying-down and rolled folds of said sheet over the entire periphery of the proximal end, the opening passing through the ring forming an entrance to the inside of the hollow body of the implant;
said distal end comprises a cup closing the distal end and secured, in a sealed manner, to the lying-down and rolled folds of said sheet over the entire periphery of the distal end of said hollow body

Another distinctive feature is that the said sheet being plastically deformable to allow the implant to expand from the folded configuration to the deployed configuration when a fluid is injected into the implant.

Another distinctive feature is that the distance between a synform fold and the next antiform fold is longer than the distance between an antiform fold and the synform fold to make it easier to roll the folds around the longitudinal axis.

According to another feature, the implant comprises, in the deployed position, a middle portion between its two ends that has the shape of a generalized cylinder, with possible and at least partial persistence of said folds, and at each of its two ends a frustoconical portion connecting the middle portion to the cup and the ring, with a permanent persistence of at least part of the lying-down and rolled folds near the ends.

According to another feature the said sheet said sheet is plastically deformable also from the folded configuration to the deployed position, in particular as a result of the persistence of the lying-down and rolled folds at the proximal and distal ends, to enable reversibility of the expansion.

According to another feature, the said cup is adapted to cooperate with the distal end of an implantation instrument passing through said implant via the opening of the proximal end, for example by virtue of at least one housing and/or protuberance complementary to at least one protuberance and/or housing of said instrument which comprises a hollow tube of which the interior canal opens into said hollow body of the implant via at least one opening allowing said fluid to be injected into the implant.

According to another feature, the said sheet is secured to the ring of the proximal end by a weld that fixes the lying-down and rolled folds against the exterior wall of said ring.

According to another feature, the said sheet is compressed around the ring of the proximal end and/or around the cup of the distal end by a second ring, said a compression ring that holds the lying-down and rolled folds against the exterior wall of said ring and/or of said cup.

Another feature is that the folds are, at least in the folded configuration, parallel to the longitudinal axis.

According to another feature, the outside diameter of said cup and/or of said ring is smaller than or equal to the maximum folded diameter of the implant.

Another distinctive feature is that the number of pairs of folds is comprised between 3 and 16, generally 4 to 12, and preferably of the order of 8.

According to another feature, the sheet is closed on itself by virtue of two folds of opposite directions (synform and antiform) formed on the two opposite edges of the sheet so as to interlock and form a longitudinal closure and impart the shape of a generalized cylinder to the sheet, at least prior to folds being produced and rolled.

Another feature is that the sheet has a thickness of between 3 and 100 microns, generally between 6 and 50, and preferably 10 and 30 microns.

According to another feature, the sheet is made of titanium alloy.

According to another feature, the sheet also comprises at least one pair of folds (a synform fold and an antiform fold) of axis not parallel to the longitudinal axis, preferably perpendicular so that the implant can expand also lengthwise, or oblique so that the implant can expand in a curved manner.

According to another feature, that the distance between the folds is variable along the circumference of the implant, so that the shape of the implant in the deployed configuration is curved or asymmetric.

According to another feature, said folded diameter is smaller than the deployed diameter by a factor of between 3 and 20, generally 3 to 8, preferably 4 to 7.

Another aim of the present application is to overcome at least some of the disadvantages of the prior art by proposing a surgical intervention system that is easy to use and allows effective stabilization of bone tissue.

This objective is achieved by an orthopedic treatment system for damaged bone tissue comprising a bone substitute cement and at least one instrument for implanting and injecting cement into the implant, characterized in that it comprises an implant according to one of the preceding claims.

According to another feature, that the instrument for implantation and for injection of cement comprises means for controlling the pressure and/or the aspiration of the cement so that the implant can be re-folded to the folded configuration if necessary.

According to another feature, that the implantation instrument is different from but complements the injection instrument the cement-injection canal of which passes through a canal inside the rod of the implantation instrument that via its distal end holds the proximal end of the implant.

Another purpose of the present application is to overcome at least some of the disadvantages of the prior art by proposing a method of manufacturing an implant according to the invention.

This purpose is achieved by a method of manufacturing an implant according to one of the preceding claims, characterized in that it comprises:
Closing the sheet on itself and welding it to form a generalized cylinder
Inserting the closed sheet on a die in the shape of a generalized cylinder having a star-shaped base, the number of branches of the star defining the number of pairs of folds of said sheet of said implant
Compressing the closed-up sheet between said die and a plurality of projecting elements of a shape that complements the hollows between the branches of the star
Rolling the folds of said sheet around the longitudinal axis
Securing said sheet to the cup and the ring.

Other features and advantages of the present invention will become more clearly apparent on reading the following description of various embodiments, with reference to the appended drawings, in which:

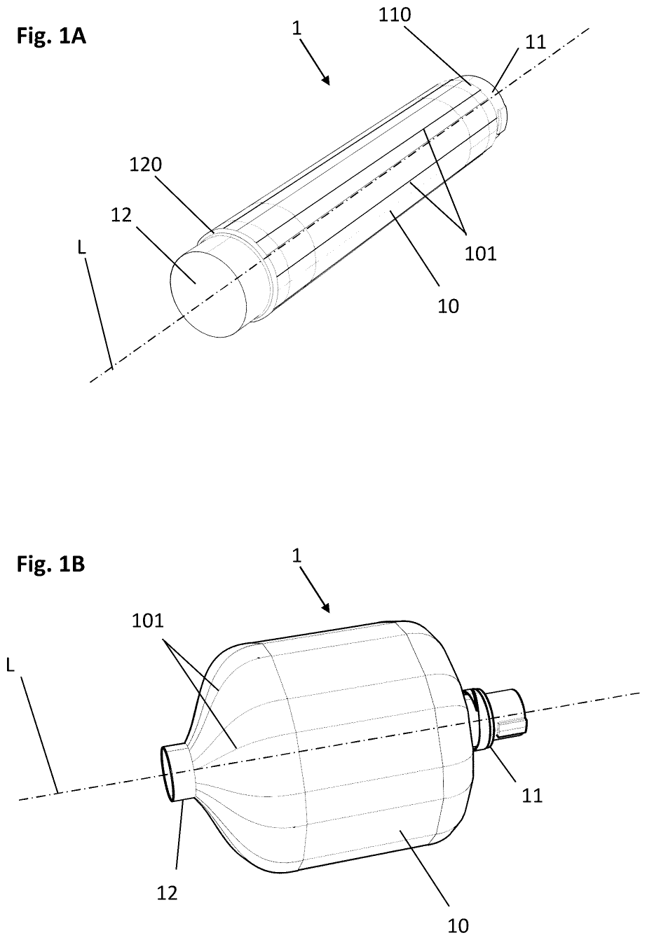

FIG. 1: FIG. 1A depicts a perspective view of an expandable implant in its folded configuration, according to certain embodiments, and FIG. 1B depicts a perspective view of the same implant in its deployed configuration;

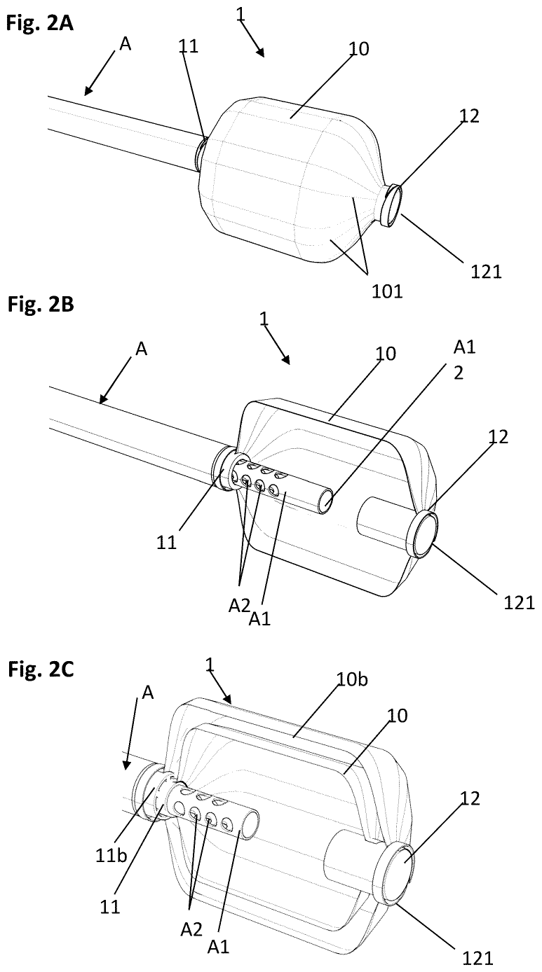

FIG. 2: FIG. 2A depicts a perspective view of an expandable implant in a deployed configuration and held by an implantation instrument, according to certain embodiments, and FIG. 2B depicts a perspective view of this same implant with a section showing the implantation instrument inside the implant and FIG. 2C shows the same view as FIG. 2B but for a double-layer implant;

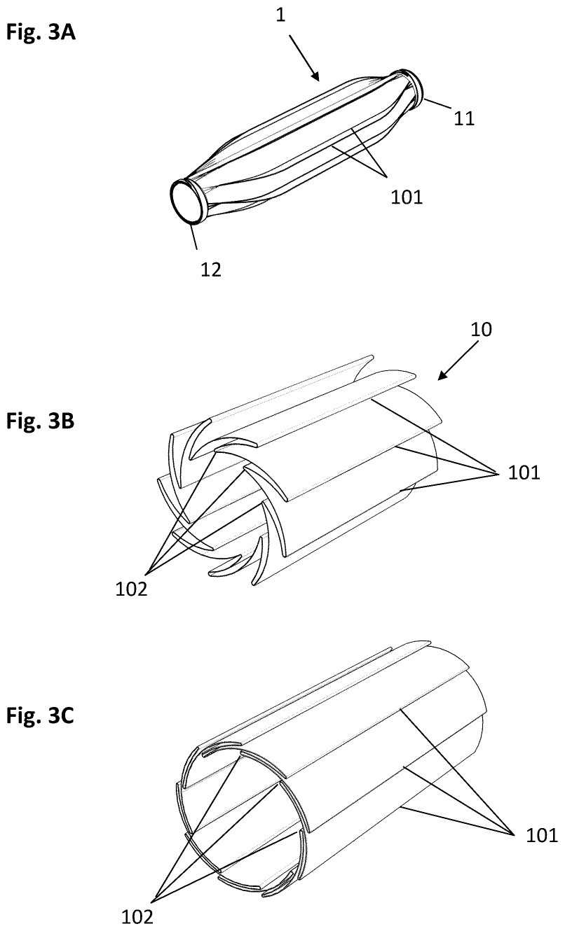

FIG. 3: FIG. 3A depicts a perspective view of an expandable implant in a semi-deployed configuration, according to certain embodiments, FIG. 3B depicts a perspective view of a sheet used for the manufacture of an expandable implant according to certain embodiments in the semi-folded configuration, and FIG. 3C depicts a perspective view of this same sheet in the folded configuration;

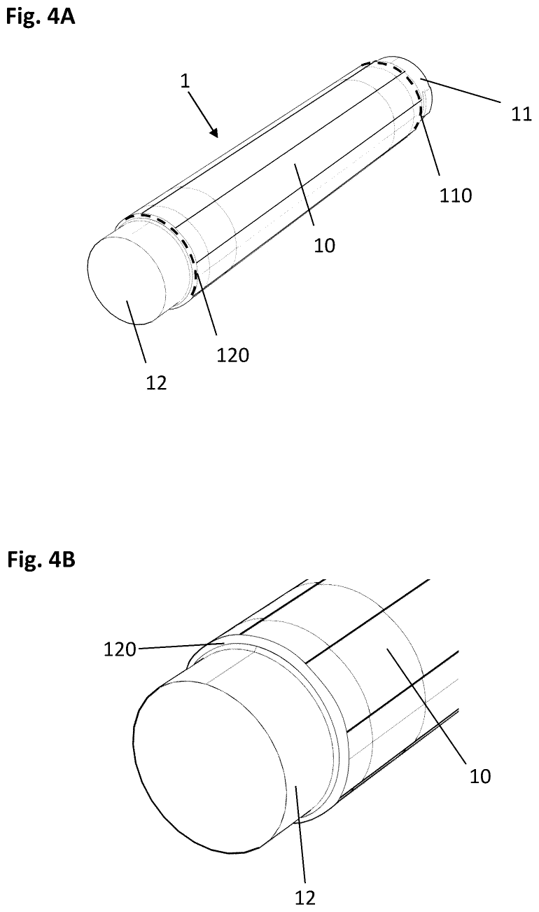

FIG. 4: FIG. 4A depicts a perspective view of an expandable implant in the folded configuration with lines of welding at the proximal and distal ends, and FIG. 4B depicts an enlargement of FIG. 4A at the distal end;

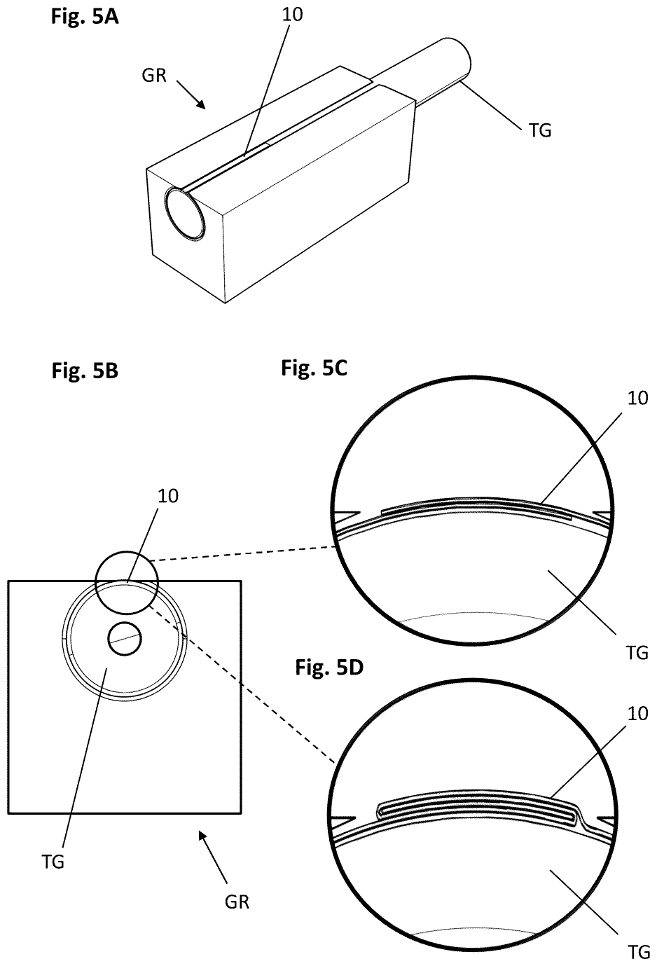

FIG. 5: FIG. 5A depicts a perspective view of a guide tool that guides the folding of a sheet of an expandable implant according to certain embodiments, the sheet being guided by means of a guide tube, and FIG. 5B depicts a profile view of this same tool with the sheet folded, the enlargements 5C and 5D depicting profile views of the overlapping of the sheet at its closure according to two different examples of embodiment;

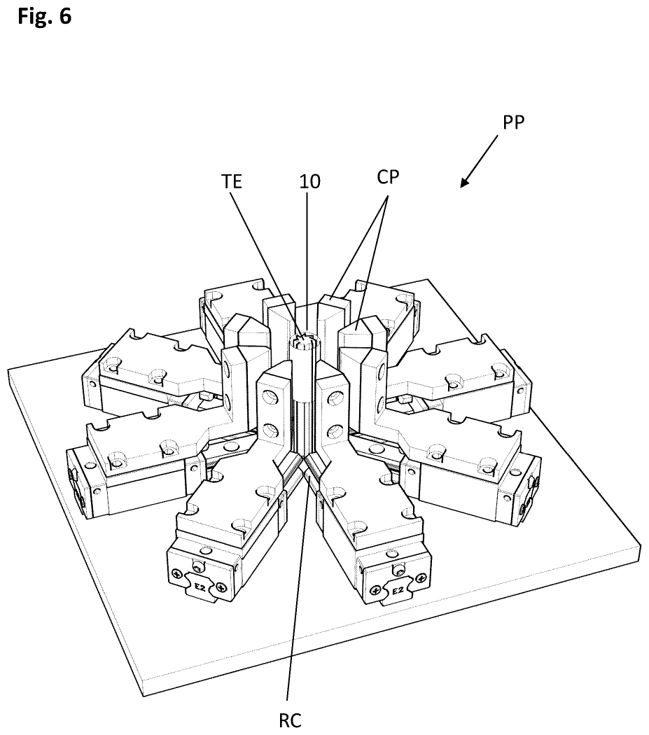

FIG. 6: FIG. 6 depicts a perspective view of a tool for the pre-folding of sheets for the expandable implant according to certain embodiments, using a pre-folding plate;

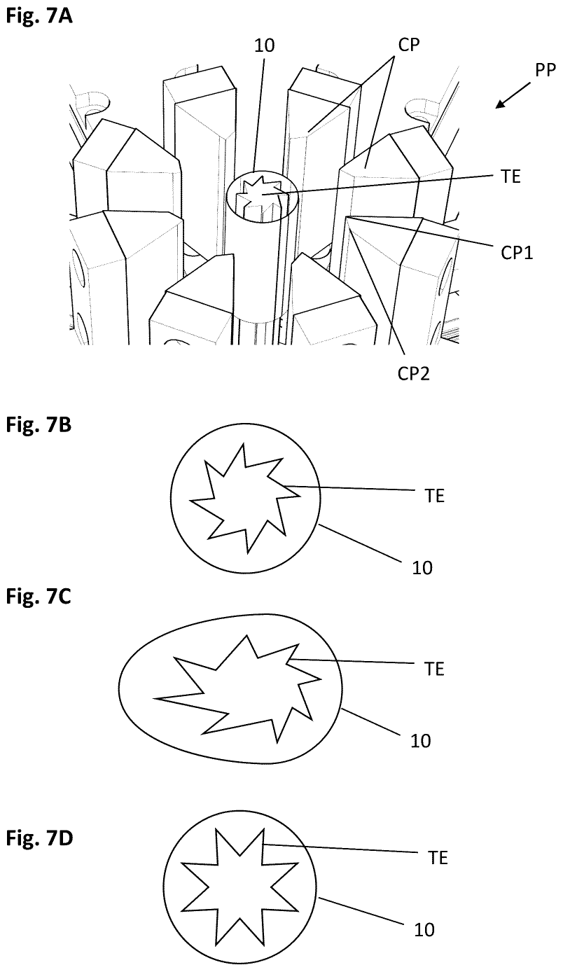

FIG. 7: FIG. 7A depicts an enlargement of FIG. 6, FIGS. 7B, 7C and 7D depict views from above of various embodiments of the pre-folding tool with a sheet of the implant slipped around a star-shaped rod of the pre-folding tool.

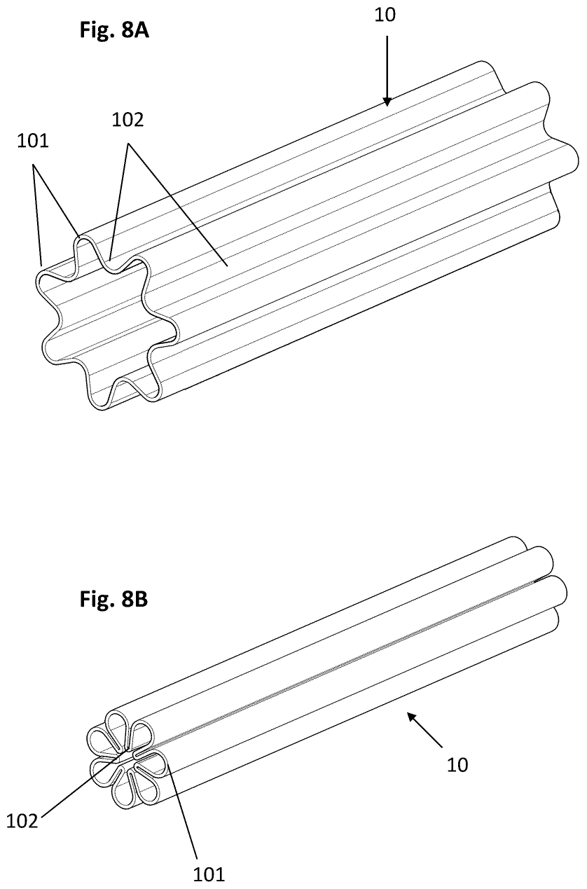

FIG. 8: FIG. 8A depicts a perspective view of a sheet that has been pre-folded using a star-shaped rod such as that of FIG. 7D, and FIG. 8B depicts this same sheet folded on itself, according to certain embodiments;

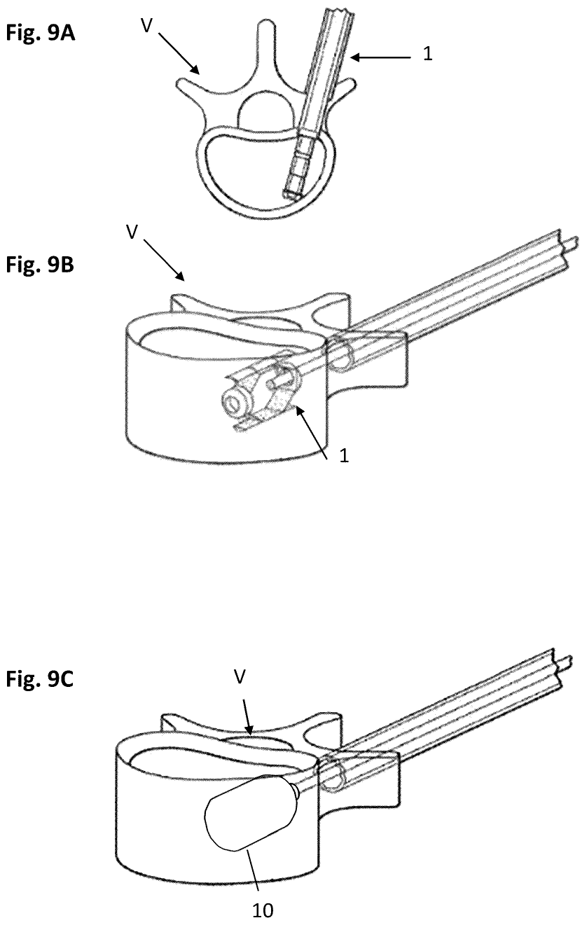

FIG. 9: FIG. 9A depicts a view from above of a vertebra in which an implant according to various embodiments is implanted, FIG. 9B depicts a perspective view of the implantation of an implant of the prior art in a vertebra, and FIG. 9C depicts a perspective view of the implantation of an implant according to certain embodiments of the present invention;

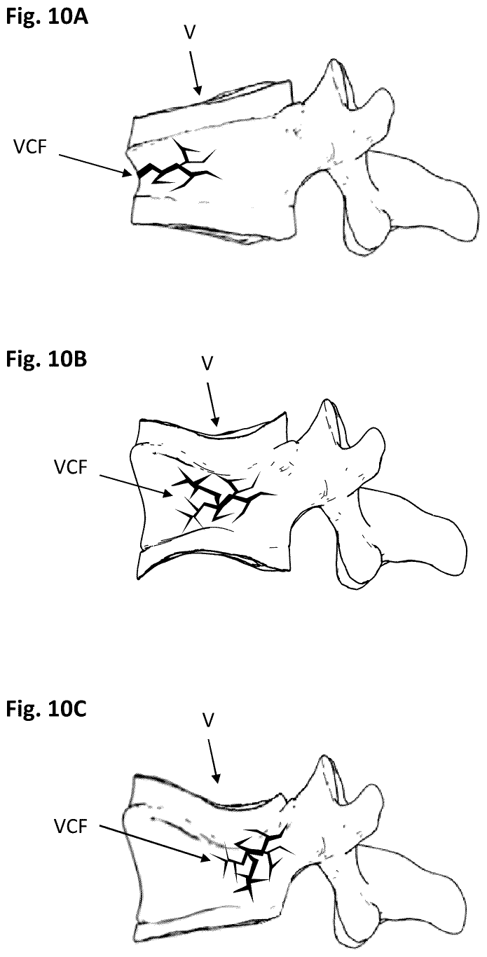

FIG. 10: FIGS. 10A, 10B and 10C depict profile views of vertebrae that have respectively suffered anterior, median and posterior vertebral compression fractures (VCFs).

The present application relates to an implant and an orthopaedic surgery system for treating fractures in bone and bone tissue in general, and to a method for manufacturing the implant. The bone implant is preferably a spinal implant, and in particular a vertebral or even in fact intervertebral implant, although other uses elsewhere than in the spine (intervertebral discs) or in other bony structures where it is necessary to fill a space left vacant as the result of a fracture (the causes of which may be various, even though they generally imply a reduction in bone density) are conceivable. Thus, vertebral compression fractures (VCFs) are a favourite application but are not the only conditions that can be treated using the present invention, and the person skilled in the art will appreciate the possibilities offered without requiring further details here. By way of other bones, mention may be made of the femur or the humerus (head), for example in the event of risk of collapse. This application relates to an implant and a veterinary orthopedic surgery system for treating fractured bones and bone tissue in general, as well as a method for manufacturing the implant. The bone implant is preferably a spinal implant, and in particular a vertebral or even intravertebral implant, but other uses are possible elsewhere in the spine (intervertebral spines) or in other bone structures where it is necessary to fill a space left by a fracture (the causes of which may vary, although they generally involve a decrease in bone density). Thus, vertebral compression fractures (VCFs) are a favorite application, but they are not the only ones that can be treated with the present invention, and those skilled in the art will appreciate the possibilities offered without further detail here. Other bones include the femur or humerus (head), for example, in cases where there is a risk of collapse. In the veterinary field, it is known that animals sometimes have bone densities that are very different from those of humans and, above all, vary greatly depending on the species and even on the breed or animals within the same species, particularly in the case of dogs, whose physical properties vary enormously from one breed to another. For example, dachshunds and similar breeds have long (tall) vertebrae but are not very wide compared to other species. It is therefore useful to have expandable implants that allow for significant expansion in height while having a reduced length, and it is clearly necessary to take into account the differences in the shapes and sizes of the bones of different species in order to adapt the therapy effectively with suitable implants. In addition, some species, such as cats, have very rigid cortical bone but more flexible cancellous tissue than other species. It is therefore also necessary to take into account the nature of the bone tissue. Finally, another notable example concerns horses, which have bones, particularly vertebrae, with a specific anatomical shape and which sometimes bear a heavy load. Depending on the activity (e.g., sports) and morphology, bone density varies and implants must be adapted to allow for expansion but also to bear loads. Thus, for a horse, it may be necessary to have implants with more load-bearing arms (at least 3 or 4) than for other species (where 2 support arms are sometimes sufficient). In the absence of support arms, the implant must have sufficient mechanical strength, thanks to its ability to withstand greater cement pressure than in other cases.

In addition, the tibial plateau is frequently subject to crushing, and the implants or systems of the present application are useful for restoring height in any type of bone crushing or collapse, for example in the distal part of the humerus or femur. On the other hand, as taught, for example, in document EP2921142, it is possible to use expandable implants as bone anchoring implants, and such use is also possible for implants such as those of the present application. In this case, the implants will be extended at their proximal end by an elongated body to which another orthopedic implant of another type or a surgical device for fixing other elements can be attached. Nevertheless, in the case of use as a bone anchor in a vascularized structure, such as a humeral or femoral head, the size of the implant will preferably be limited in relation to the bone structure in order to preserve vascularization and promote bone healing.

Certain embodiments provide for the injection of a fluid (e.g. “bone cement”, which is generally based on a polymer such as PMMA, for example, and is well known to those skilled in the art, and therefore no detail on the cement will be given here). Thus, once positioned, the implant can be stabilized by such an injection of cement. However, because cement leakages are a major problem in this field, various embodiments propose containing the cement in a fluidtight casing, the post-injection volume of which can be controlled by virtue of the structure and the material of the casing, as a function of the injected pressure (and the configuration of the bone tissue, preferably assessed in advance, as is the general practice in this field). Fluidtightness is of course relative and this term is not limiting either, since the level of fluidtightness is in fact adapted to the viscosity of the cement at the moment of its injection. Certain embodiments in particular allow a proportional expansion of the casing as a result of the (relative) flexibility of the sheet (10) of biocompatible metallic material. This material is generally a titanium alloy obtained in the form of a very thin sheet, preferably by rolling to give a controlled surface condition and a controlled thickness, notably a thickness comprised between 3 and 100 microns, generally between 6 and 50 and preferably 10 and 30 microns. In general, the present invention uses at least one sheet (10) of biocompatible metal or biocompatible metal alloy, such as titanium or its alloys, particularly with nickel or others, but also nitinol or stainless steel or their alloys. Advantage is taken of recent techniques for obtaining very thin sheets of such metals, in particular with a thickness of less than 50 or even 40 μm, which makes it possible to obtain relatively flexible and elastic sheets, but above all, the plastic deformation of which can be used reversibly without reaching their tear limit, by creating folds arranged longitudinally on the implant. In particular, it is possible to provide a maximum unfolded volume that is greater than the volume required for the desired applications, so that this limit is never reached and it is possible to fold and then unfold the implant, even several times (for example, in the event of incorrect positioning of the implant), without the risk of uncontrolled tearing and leakage. Thus, thanks to this type of sheet and the configuration of their interlocking folds, it is possible to obtain expansion ratios between the folded volume and the deployed volume ranging from 2 to 20, or even 30, and it is also possible to control the shape of the implant in the deployed configuration, depending on the arrangement of the folds, in the manner of origami. Finally, although the main goal here is to prevent cement leakage, it can sometimes be useful to control the release of cement outside the implant, so that it is no longer a question of leakage but of controlled release, for example to allow adhesion to certain surrounding structures (usually bone structures). Similarly, as the injected fluid is not necessarily cement (or at least not the fluid that would come out of the implant), it may in fact be useful to administer molecules through such controlled release of this fluid. Thus, various embodiments provide for a certain porosity of the sheets (10) at least in certain portions of the implant, for example through holes of controlled microscopic size and controlled number and density. In any case, this sheet is capable of reversible plastic deformation a number of times that is satisfactory for the target application since it notably offers the possibility of retracting the casing formed by the sheet in the event of a problem (biocompatibility and resistance to tearing). Specifically, in general, controlling the metering of the cement allows monitoring for the fifteen minutes of polymerization time during which it is possible to retract the casing and aspirate the cement. Furthermore, through the injection of cement and the expansion of the casing, the implant fills the spaces in the damaged tissues according to the compression and bone-resistance forces relative to the hydraulic pressure supplied during the injection of cement. From such a sheet, it is necessary to obtain a closed structure, which already means that the sheet needs to be closed on itself and locked in position. To do that, welding (or bonding or brazing, these terms being nonlimiting here) may be used to join together two superposed edges or edges with interlocking turn-ups, to facilitate the welding and make it more robust. Certain embodiments therefore envisage closure by welding from the outside, which is simpler and more robust because of the superposition of layers at these complementary folds.

Various embodiments make it possible to obtain an expandable implant with very small dimensions in the folded configuration while at the same time guaranteeing a satisfactory volume in the deployed configuration. Thus, the passage required to introduce the implants of the present application is generally smaller than that of the known implants, whereas the expansion is greater than that of these known implants. Specifically, the folded volume or diameter is smaller than the deployed diameter by a factor of between 3 and 20, generally 3 to 8, preferably 4 to 7. This ratio depends of course on the amount of cement injected and certain embodiments take advantage of the fact that it is possible to provide an implant that is capable of deploying more than necessary, in particular by retaining folds in the deployed configuration. Thus, the volume of the implant will be determined based on the reduced size required for the introduction into the bone tissue and therefore with reference to the folded volume. However, different volumes are provided for the deployed configuration, since the number of folds and the length of the folds make it possible to increase the deployment ratio.

The term “secured” here means the two elements are secured to one another, either permanently (or near-permanently) but also sometimes that a connection is made so that one element can be actuated by another. Thus, screw-fastening or collaboration between shapes for temporarily locking the elements together are covered by this nonlimiting term.

The terms “ring”, “sleeve”, or “tube” refer to hollow structures such as bands, conduits or pipes, but nonlimitingly, notably having various shapes (on the inside as on the outside), although a cylindrical shape is preferred. The term “canal” by contrast is preferably used here to refer to a passage rather than to the element that contains it, and the term “opening” here refers to the fact that an element is open and able to be passed through, opening out into another structure or another element. In general, the terms “sleeve”, “tube” or “conduit” refer to longer elements than rings or bands, although their use here is likewise nonlimiting. Furthermore, the terms “socket” or “cup” refer also to hollow structures that are open at one end but closed at the other end, such as plugs, closures, constrictions or restrictions, and these terms are used indiscriminately without any limitation.

The term “hinge” is used here in its functional sense without implying any structural limitation, and may in fact refer to mechanical hinges even though these are preferably formed (as illustrated in the non-limiting examples in the figures) by thinning (or narrowing, removing material from) elements such as the support arms or other elements. Thus, a hinge is in fact an articulation point or region since it is known in the art that there is generally no danger associated with providing such pivot mechanisms in implants because the materials of which they are made are suited to this type of articulation.

The terms “antiform fold”, referred to as “convex”, and “synform fold”, referred to as “concave”, are used by analogy with the definitions of folds in numerous technical fields, including that of geology, but it will be appreciated that “convexity” is defined here with respect to the outside of the implant. An antiform or convex fold is therefore a fold that turns the material inwards, while an antiform fold turns the material outwards. The succession of the two types of folds makes it possible to limit as far as possible the volume that is folded. In addition, certain embodiments envision a succession of long folds and of short folds making rolling and/or compaction easier by limiting the extent to which material is superposed in the folded configuration. It will also be noted that the number of folds is not limiting either but that it instead makes it possible to maintain the irregularity or trueness of shape of the implant as it deploys, this likewise offering advantages, in particular in terms of stabilization. In addition, it is still preferable to have an even distribution of surface areas between the folds for uniform deployment allowing uniform deployment, although the invention also envisions other applications and notably folds of different sizes depending on the region of the implant, so as to obtain asymmetric deployment and better therapeutic outcomes. Moreover, the present invention makes it possible to control the shape of the implant once deployed by also setting the distance between the folds. Specifically, the distance between the synform/antiform folds, and therefore the distance between the long folds and the short folds, governs the way in which it deploys. Advantageously, if the density is greater at one point on the periphery, deployment will be greater and if it is lower, the casing will be able to deploy to a lesser extent. It will be appreciated that asymmetry and curving is thus obtained by more extensive deployment in regions that have the greatest folds content. Likewise, it is possible to envisage more material (a larger surface area of sheet) on one side for example so the lateral expansion will be greater on this side than on the other. Moreover, in certain embodiments, the sheet is welded to the flanges and is therefore unable to deploy further than the distance between the flanges, which distance will have been set by the lever mechanism. What is thus obtained is an implant the expansion of which is limited in one dimension (in general the essential dimension in which a precise height or width is to be restored) but not in another dimension, so that the injection of cement will deploy the casing into the volumes of low bone density that may be present around the implant. It will also be noted that the fluid injection instrument may be fitted with means for controlling the injected pressure (a pressure gauge, for example) and for indicating the resulting volume so that the expansion into the bone tissue can be controlled effectively. Finally, it will be appreciated that the instrumentation proposed in the present application in certain embodiments, using an implant holder (or ancillary) of relatively conventional type to hold the implant and introduce it into the bony tissue, but also of less-conventional type for expanding it into the bony tissue, also offers the advantage that all the implantation and stabilization steps can be carried out using just one single instrument and in a continuous operation. Specifically, the ancillary with a hollow tube for conveying cement through the tube that holds the cement makes it possible to offer an instrument that allows the surgical intervention to be performed quickly and efficiently. After drilling, the implant is introduced and, without withdrawing the instrument, the casing can be inflated with the cement and then the tool can be withdrawn before, during or even after the polymerization of the cement (for example using a mechanism for cutting the hardened cement as the instrument rotates). The time taken to perform the surgical operation is of course markedly reduced as also the stability of the implant which is not released at any time until it has been stabilized by the injection of cement filling all the free volumes around it, unlike in certain solutions of the prior art.

The terms “cylinder”, “cylindrical” or “generalized cylinder” are used in the present application indiscriminately to make the invention easier to explain and in fact all refer to a “generalized cylinder”, which is to say a three-dimensional shape defined by a height (parallel to the longitudinal axis) and two bases (transverse to the longitudinal axis) which may have any shape whatever, even though a circular shape is preferred in order to simplify the manufacture and limit the risk of lesion of the tissue into which it is introduced. Preferably, this “cylinder” is a right cylinder, which is to say that its bases are aligned with respect to the generatrix (or height) of the cylinder. Furthermore, because the implant may deploy in a tissue to conform to the shape of the space into which it has been introduced (modifying this thanks to the pressure it exerts on this tissue), it is possible for the shape not to be constant so the two bases of the cylinder may have different shapes (areas).

As a result, the term “diameter” is used, in the present application, to refer in fact to the longest dimension of the generalized cylinder transverse to the height (or longitudinal axis) thereof, namely in a plane (referred to as “transverse”) parallel to that of the bases of such a generalized cylinder. Thus, the term “diameter” may in fact refer to the length of the diagonal of a square or of a rectangle or else (for any arbitrary shape) to the longest distance between two points included in such a transverse plane and lying on the circumference of such a cylinder. Likewise, the terms “circumference”, “periphery” or “perimeter” are used here to refer to the perimeter of these bases of any arbitrary shape.

Likewise, the terms “conical” or “frustoconical” are used here to denote shapes that widen from a minimum “diameter” (or surface-area/surface) up to a maximum “diameter”, but they do not imply any limitation as to the shape of the parameter which may or may not be circular.

In general, the present application relates to an expandable bone implant (1) for veterinary orthopaedic surgery for restoring the volume and/or geometry of a bone by expansion between a folded configuration and a deployed configuration, said implant comprising a hollow body extending along a longitudinal axis (L) between a proximal end (11) adapted to cooperate with an implantation instrument (A) for holding the implant and a distal end (12) intended to be first inserted into the bone. This proximal end can be connected to a gripping instrument (known as an implant holder) and is therefore capable of cooperating with the latter by means of attachment or physical connection, for example, as known to those skilled in the art. Nevertheless, certain embodiments provide specific and advantageous attachment means to facilitate the gripping of the implant by an implant holder and, above all, release of the implant by an L-shaped movement of the implant holder. Furthermore, by being connectable to the instrument, the implant can generally be actuated for expansion (in this case by injecting a fluid inside), as is widely known in the prior art. Indeed, many systems comprise expandable implants that can be actuated when mounted on an implant holder that includes an actuating means for expanding the implant (generally a conduit and/or a rod passing through the implant holder to open into a cavity in the implant and/or cooperate with an implant component that allows its expansion, the actuation generally involving a pushing and/or pulling force). The skilled person will therefore understand from reading this application that the implant can be defined without further detail on the instrument and the actuation, since these are conventional mechanisms in the field and the system comprising the implant and the instrument is of course fully defined, but that the implant alone is in fact also well defined in its actuatable nature. mechanisms in the field and that the system comprising the implant and the instrument is of course fully defined, but that the implant alone is in fact also well defined in its operable nature independently of the instrument and without unnecessary detail on the actuation mechanism (sliding rod, for example) insofar as these are perfectly standard or conventional mechanisms in the field. It is understood in the field of the present application that the term “actuable” implies pushing or pulling, and the present application thus provides sufficient explanation for the implant to be considered sufficiently clearly defined without additional reference to the instrument enabling its actuation. On the other hand, certain embodiments may relate to the instrument itself, through the originality of its elements allowing the implant to be grasped and/or actuated for expansion, and these characteristics then define the instrument independently of the implant, since they do not require any particular details about the implant other than those relating to the function performed by the instrument.

Such an implant (1) is preferably characterized in that
the wall of said hollow body is formed by a sheet (10) made of a biocompatible metal alloy, closed on itself in a sealed manner, between said proximal (11) and distal (12) ends;
said sheet (10) has, at least in the folded configuration, a plurality of pairs of folds, each of the pairs comprising an antiform fold (101), referred to as convex, and a synform fold (102), referred to as concave, said folds lying on top of each other in the folded configuration so that the surfaces present between each of said convex and concave folds are rolled around the longitudinal axis (L);
said proximal end (11) comprises a sleeve or ring secured, in a sealed manner, to the lying-down and rolled folds of said sheet (10) over the entire periphery of the proximal end (11), the opening passing through said sleeve forming an entrance to the inside of the hollow body of the implant (1);
said distal end (12) comprises a socket closing the distal end (12) and secured, in a sealed manner, to the lying-down and rolled folds of said sheet (10) over the entire periphery of the distal end of said hollow body;
said sheet (10) being plastically deformable to allow expansion of the implant from the folded configuration to the deployed configuration, when a fluid is injected into the implant (1) through said sleeve.

In certain embodiments, the distance between a synform fold and the next antiform fold is longer than the distance between an antiform fold and the synform fold to make it easier to roll the folds around the longitudinal axis (L). To make it easier to roll the sheet (10) on itself and obtain a smaller folded volume, it is preferable to envisage an alternation of long folds and short folds. To do that, it is possible to use pre-folding cams (CP) having two edges at different angles, with a star-shaped rod (TE) likewise of asymmetric shape complementing the first angle (CP1) of the pre-folding cam and the second angle (CP2) of the pre-folding cam, as for example depicted in FIGS. 6, 7A, 7B and 7C, but it is also possible to have pre-folding in a symmetrical shape, as for example depicted in FIGS. 7D, 8A and 8B, although these embodiments gave less-advantageous folding than asymmetric folding with an alternation of long folds and short folds.

In general, it is understood that the implant will retain, even when deployed, at least some flattened and rolled folds near the proximal and distal ends, but the dimensions and strength properties of the sheet (10) used allow the implant to be obtained and ensure that these persistent folds do not interfere with function or cause mechanical or physiological problems in the bone tissue. In certain embodiments, in the deployed position, the implant comprises a middle portion between its two ends that has the shape of a generalized cylinder, with possible and partial persistence of said folds, said middle portion being extended, at the end corresponding to the proximal end (11), by a frustoconical portion connecting the middle portion to said sleeve and, at the end corresponding to the distal end (12), by a frustoconical portion connecting the middle portion to said socket, the frustoconical portions having a permanent persistence of at least part of the lying-down and rolled folds near the proximal end (11) and distal end (12).

In certain embodiments, said sheet is plastically deformable also from the folded configuration to the deployed configuration, in particular as a result of the persistence of the lying-down and rolled folds at the proximal and distal ends, to enable reversibility of the expansion.

In certain embodiments, said socket is adapted to cooperate with the distal end of an implantation instrument (A) passing through said implant via the opening of the proximal end (11), for example by virtue of at least one housing and/or protuberance complementary to at least one protuberance and/or housing of said instrument (A) which comprises a hollow tube (A1) which is adapted to pass through said sleeve and of which the interior conduit opens into said hollow body of the implant (1) via at least one opening (A2) allowing said fluid to be injected into the implant (1).

In certain embodiments, said sheet is secured to said sleeve of the proximal end (11) by a weld (110) that fixes the proximal end of the lying-down and rolled folds against the exterior wall of said sleeve and/or secured to the cup of the distal end (12) by a weld (120) that fixes the distal end of the lying-down and rolled folds against the exterior wall of said socket.

In certain embodiments, said sheet is compressed around the ring of the proximal end (11) and/or around the cup of the distal end (12) by a second ring, said compression ring that holds the lying-down and rolled folds against the exterior wall of said sleeve and/or of said socket.

In certain embodiments, the folds are, at least in the folded configuration, parallel to the longitudinal axis (L). In certain embodiments, the outside diameter of said socket (10) and/or of said band (11) is smaller than or equal to the maximum folded diameter of the implant. In certain embodiments, the number of pairs of folds is between 3 and 16, generally 4 to 12, and preferably of the order of 8. However, 2 or 3 folds may sometimes suffice, particularly for small implants for small animals or small bones.

But the greater the number of folds, the less the material will deform, the less risk there will be of tearing, and the easier it will be to unfold. Thus, it is possible to plan for up to 20 folds, even for medium-sized animals, and when it comes to large animals such as horses, it is possible to allow for 25 to 30 folds, or even more.

In certain embodiments, the sheet (10) is closed on itself by virtue of two folds of opposite directions (synform and antiform) formed on the two opposite edges of the sheet so as to interlock and form a longitudinal closure and impart the shape of a generalized cylinder to the sheet (10), at least prior to folds being produced and rolled.

In certain embodiments, the sheet (10) has a thickness of between 3 and 100 microns, generally between 6 and 50, and preferably 15 and 30 microns. In certain embodiments, the sheet (10) is made of titanium alloy.

In certain embodiments, the sheet also comprises at least one pair of folds (a synform fold and an antiform fold) of axis not parallel to the longitudinal axis (L), preferably perpendicular so that the implant can expand also lengthwise, or oblique so that the implant can expand in a curved manner.

In certain embodiments, the distance between the folds is variable along the circumference of the implant, so that the shape of the implant in the deployed configuration is curved or asymmetric.

In certain embodiments, said folded diameter is smaller than the deployed diameter by a factor of between 3 and 20, generally 3 to 8, preferably 4 to 7.

The present application also relates to a system for orthopaedic treatment of damaged bone tissue, comprising a bone substitute cement and at least one instrument (A) for implantation and for injection of cement into the implant, characterized in that it comprises an implant according to various embodiments.

In certain embodiments, the instrument for implantation and for injection of cement comprises means for controlling the pressure and/or the aspiration of the cement so that the implant can be re-folded to the folded configuration if necessary.

In certain embodiments, the implantation instrument is distinct from but complements the injection instrument of which the cement-injection canal passes through a canal inside the rod of the implantation instrument that via its distal end holds the proximal end of the implant.

The present application also relates to a method for manufacturing an implant according to various embodiments, characterized in that it comprises:
Closing the sheet on itself and welding it to form a generalized cylinder Inserting the closed sheet on a die in the shape of a generalized cylinder having a star-shaped base, the number of branches of the star defining the number of pairs of folds of said sheet of said implant
Compressing the closed-up sheet between said die and a plurality of projecting elements of a shape that complements the hollows between the branches of the star
Rolling the folds of said sheet around the longitudinal axis Securing said sheet to said socket and the ring.

For example, rolling may be achieved by introducing the closed and pre-folded sheet into a conduit the diameter of which narrows progressively down to the desired diameter for the implant, by sliding and twisting the implant in this conduit (for example with a guide inside the sheet to prevent it from becoming crushed).

The act of securing to the sleeve and the socket will generally be achieved using welding (120), preferably with the sheet being crushed beforehand onto the circumference of the socket or the sleeve, for example by means of a compression ring (121), examples of which are depicted in certain figures. Specifically, while it is possible to weld directly, compressing the folds in place remains preferable.

The illustrative and nonlimiting figures of the present application will now be described in detail the better to explain the various embodiments and provide examples of structural elements that can be used in the foregoing context. That which follows must not be considered as being limiting since the various elements or components illustrated are merely examples and the figures may combine elements or components that are not necessarily dependent on one another.

FIG. 1A depicts a perspective view of an expandable implant in its folded configuration, according to certain embodiments, with a seal (110) proximal to the sheet (10) on the proximal end (11) of the implant (1) and with a seal (120) distal to the sheet (10) on the distal end (12) of the implant (1), and FIG. 1B depicts a perspective view of the same implant in its deployed configuration, but with the proximal seal formed by a split ring, as described later in the present application.

FIG. 2A depicts a perspective view of an expandable implant in the deployed configuration and held by an implantation instrument, according to certain embodiments, and FIG. 2B depicts a perspective view of this same implant with a section showing the implantation instrument inside the implant. FIG. 2B specifically depicts a perspective view with a partial section of the casing of the implant deployed by the cement injection, during withdrawal of the injection instrument by sliding its cannula (A1) into the cannula (A) of the insertion instrument (implant holder) which holds the proximal end of the implant (1). The distal end of the cannula (A1) cooperates with a distal cup of the implant (1) during implantation to retain the distal end (12) and inject cement via holes (A2) opening from the inside of the cannula (A1) to the inside of the casing. When the injection cannula (A1) is withdrawn, at the end of deployment, the holes (A2) are closed by the inside of the cannula (A) of the implant holder (A) and the distal end of the injection cannula (A1) preferably comprises a plug (A12) which then ensures at this time a discontinuity between the inside of the injection cannula (A1) and the inside of the implant, facilitating the polymerization of the cement without it spreading into the instrumentation, thereby facilitating the release of the implant, preferably once the cement has polymerized. If the end is open without such a plug (A12), the cannula then needs to be designed to be breakable and optionally provided with means for cutting or breaking the polymerized cement it contains when the implant is released into the implantation site. Specifically, in certain embodiments, the distal end of the cannula (A1) is open so that the cement can be injected through this end, and the holes (A2) then can be omitted, but it is still preferable for such holes (A2) to be used to allow the cement to exit laterally inside the implant, around the circumference of the cannula (A1) and over a significant portion of the size of the implant.

In certain embodiments, the implant may comprise a second sheet (10b) surrounding the first sheet, made from the same material or another material, to form a double casing, for example as depicted in FIG. 2C. Such a double casing may offer numerous assorted advantages, particularly of providing thermal insulation to protect the tissue from the heat of polymerization (thanks for example to a fluid that limits the transmission of heat) or simply of providing additional safety to ensure that cement does not leak out if one of the sheets becomes torn. In that case, at least one of the ends of the implant, particularly the proximal end (11), may comprise an additional ring or cup concentric with the first ring or with the first cup or, for example as depicted in FIG. 2C, a double-channel ring or double ring, to secure this second sheet (10b) while maintaining a space between it and the first sheet (10), but it is possible to join these two sheets (10, 10b) together at the ends. In the case of two more widely spaced sheets, it is possible to provide an injection inlet, for injecting, between the two sheets (10, 10b), a fluid different from or identical to the first, for example by means of a double ring or a single ring with two conduits.

Such a double ring may, for example, comprise spacers between a first ring and a second ring (11b) which is concentric with the first ring to form between them an annular conduit allowing the injection of this second fluid (such as for example a lubricating fluid that improves the sliding of one sheet relative to the other and thus facilitates deployment). Other arrangements are possible, provided that they include a duct opening into the envelope formed by the first sheet and a duct opening into the space between the two sheets. During manufacture, these two sheets can then be folded and rolled at the same time or successively. but their welds (or crush joints), between themselves and/or to the ring and/or to the crucible, shall be made successively to ensure that the space between them is maintained. These double-sheet embodiments enable the injection site to be preformed (by compressing spongy bony tissue) but can also enable, for example, said fluid to be injected in two stages for better adjustment of the shape, of the resulting temperature in the tissue and/or of the rate of polymerization of the fluid (for example by adjusting the mix of the compounds of the cement). On the other hand, since it is possible to use a second fluid other than cement inside, it is possible to use the compartment between the two sheets as a cooling circuit by circulating a fluid during the polymerization of the cement, so as to protect the tissue from the heat produced during said polymerization. Such a double-sheet implant (1) therefore requires a double cannula comprising two concentric conduits or parallel each opening into one of the spaces formed, as the person skilled in the art will appreciate from FIG. 2C without further explanation being required.

FIG. 3A depicts a perspective view of an expandable implant in a semi-deployed configuration, according to certain embodiments, FIG. 3B depicts a perspective view of a sheet used for the manufacture of an expandable implant according to certain embodiments in the semi-folded configuration, and FIG. 3C depicts a perspective view of this same sheet in the folded configuration.

FIG. 4A depicts a perspective view of an expandable implant in the folded configuration with a line (110) of welding at the proximal end (11) and a line (120) of welding at the distal end (12), and FIG. 4B depicts an enlargement of FIG. 4A at the distal end, showing the distal cup that closes off the distal end of the implant.

FIG. 5A depicts a perspective view of a guide tool that guides the folding of a sheet of an expandable implant according to certain embodiments, the sheet being guided by means of a guide tube, and FIG. 5B depicts a profile view of this same tool with the sheet folded, the enlargements 11C and 11D depicting profile views of the overlapping of the sheet at its closure according to two different examples of embodiment. In order to obtain a sheet (10) that is folded on itself in the shape of a cylinder, use is preferably made of an internal guide (TG) as for example a guide tube (but potentially a roller of which the relative position with respect to the sheet is preferably able to move parallel to the longitudinal axis). This guide allows the sheet (10) to be rolled up on itself and introduced into an external guide (GR), as for example a folding guide comprising a conduit to accept the folded sheet as illustrated for example in FIG. 5A, or an external roller preferably able to move in a manner complementary to the internal guide, so as to enable welding as the rollers gradually progress along the longitudinal axis that corresponds to the height of the cylinder. Guiding the sheet using at least one of the internal and external guides enables the two edges (or ends) of the sheet (10) initially of rectangular shape and bent round on itself to be positioned in such a way that two of its edges are partially superposed. These edges positioned one above the other may then be welded together, for example as depicted in FIG. 5C, but it is possible to create 2 folds in opposing directions on each of these two edges of the sheet in order to obtain interlocked folds, for example as depicted in FIG. 5D, this making the welding easier particularly by limiting the risks of holing the sheet and/or making it possible to improve the reliability and stability of the implant particularly at the time of its subsequent deployment.

FIG. 6 depicts a perspective view of a tool for the pre-folding of sheets for the expandable implant according to certain embodiments, using a pre-folding plate. Such a pre-folding plate has a rod of which the cross section is in the shape of a star, referred to as a star-shaped rod (TE), on which it is possible to put the sheet closed on itself in the form of a cylinder, in order to partially crush (deform, pre-fold) this sheet against the star-shaped rod by means of cams comprising one or more ridges matching the exterior shape of the star-shaped rod. This FIG. 6 illustrates a preferred embodiment of a tool enabling the creation of an alternation of long folds and short folds or an alternation of symmetrical folds depending on the shape of the ends of the pre-folding cams (CP) that collaborate with a complementing star-shaped rod (TE) for pre-folding the sheet between the rod and the cams. Specifically, certain embodiments of such a tool have rails (RC) bearing pre-folding cams (CP) which may have two edges (with two ridges) having angles that are different (with respect to the sliding axis of the cams). Thus, a first cam angle (CP1) and a second cam angle (CP2) are provided which are different so as to obtain long folds and short folds, whereas cams (CP) with a single ridge and cam angles that are identical from one cam to the other make it possible to obtain symmetrical folds. The folding cams, by sliding along their respective cam rail, enable the sheet (10), previously closed on itself in cylindrical form, to be deformed (e.g. crushed) and pre-folded. To do that, this sheet (10) is inserted over a star-shaped rod (TE) the cross section of which has the shape of a star with asymmetrical branches in the case of long folds and short folds, or with symmetrical branches in the case of symmetrical cams and folds, as illustrated in FIGS. 7B and 7D respectively.

FIG. 7A depicts an enlargement of FIG. 6, FIGS. 13B, 13C and 13D depict the views from above of various embodiments of the pre-folding tool with a star-shaped rod and a sheet of the implant slipped around it. In the enlargement of part of FIG. 6 that is FIG. 7A, it is easier to see how the pre-folding cams and the star-shaped rod collaborate. FIG. 7C depicts a variant embodiment in which the star-shaped rod has branches the dimensions of which vary around the circumference of the star-shaped rod, which means that the corresponding cams will have shapes that differ from one cam to another, whether for obtaining long folds and short folds as in the example depicted, or symmetrical folds, so that the folded sheet in the form of an asymmetric flattened cylinder will, once pre-folded by this tool, have an asymmetrical shape that it will maintain also once deployed.

FIG. 8A shows an example of a folded sheet in the form of a cylinder that has been pre-folded with symmetrical folds, and FIG. 8B shows this same sheet condensed in on itself to reduce its diameter, for example to reduce it as far as possible until its internal folds (102) are adjacent to one another. It will be noted that in such an embodiment it is also possible to reduce the diameter of the casing formed by the sheet, for example by introducing it into a tapering tube or some other means for compressing the periphery of the sheet and notably the external folds (101). FIG. 9A depicts a view from above of an instrument for inserting an implant into a vertebral body according to the prior art and/or the present invention, and FIG. 9B depicts a perspective view of such an insertion of an implant of the prior art, whereas FIG. 9C depicts a perspective view of the implantation of an implant according to the present invention, once deployed inside the vertebral body.

It will be noted that the tool depicted in FIG. 6 is not necessarily necessary and that other folding means can be envisaged, although this tool is particularly effective and reliable in terms of the results obtained. In addition, it will be noted that synchronous sliding between the various folding cams on their respective rail is preferable and such synchronous sliding can be obtained, for example, by electronic control of the sliding of the cams on their rail. Furthermore, it is possible to carry out this folding in the absence of synchronicity between the cams but it is preferable when the folds are made successively in the same direction, tangentially to the circumference of the sheet, and regularly in order to avoid defects in folding and deformation of the sheet, which is relatively flexible but sometimes remains fragile for instances of deformation that are located too closely. It will also be noted that in the case of symmetrical folds, as for example in FIGS. 8A and 8B, the small amplitude of deployment, as illustrated for example in FIG. 8B, may nevertheless be advantageous at times since it makes it possible to limit the risks of undesired deformation of the bone tissues during deployment of the implant. Specifically, the flexibility of the sheet allows adaptation to the anatomical shape in order to restore a physiological geometry of the bone tissues (whether vertebral or not), however, the cement injection pressure is generally of a value that may allow excessive deformation of the tissues and control of the final volume of the implant is therefore preferable, as for the amplitude of deployment. Thus, the ratio of the folded volume to the deployed volume is an important advantage obtained by the folding (of the origami type) in the present invention, which makes it possible to ensure the final volume, but also the shape of the implant once deployed. The use of symmetrical or non-symmetrical folds or combinations thereof around the circumference of the implant makes it possible to provide an optimized (or even tailor-made) final shape to fill the space necessary for the geometric restoration of the structure.

FIGS. 10A, 10B and 10C depict profile views of a vertebra fractured in the anterior portion, in the median portion and in the posterior portion, respectively. The invention allows these kind of vertebral fracture to be treated by arranging the deployable implant in the correct position in the plane of the implantation site, using antero-posterior and/or medio-lateral positioning and adjusting the depth of insertion and/or the angle of insertion of the implant, according to the type of surgical approach being used (for example lateral, anterior, dorsal, transforaminal, transpedicular, etc.).

The present application describes various technical and advantageous features with reference to the figures and/or to various embodiments. The person skilled in the art will appreciate that the technical features of one given embodiment may in fact be combined with features of another embodiment unless the contrary is explicitly mentioned or unless it is obvious that these features are incompatible or that combining them will not provide a solution to at least one of the technical problems mentioned in the present application. In addition, the technical features described in one given embodiment may be taken in isolation from the other features of this embodiment unless the contrary is explicitly mentioned.

Detailed list of references in the figures:
1 implant
10 sheet
10b second sheet
101 antiform fold
102 synform fold
11 proximal end
11b double ring
110 proximal weld
12 distal end
120 distal weld (fluidtight connection)
121 compression fixing (e.g. split ring)
A implantation instrument
A1 hollow tube
A2 opening of the hollow tube
A12 distal plug
TG internal guide
GR external guide
PP pre-folding plate
TE star-shaped rod
PP pre-folding plate
CP pre-folding cam
CP1 pre-folding cam first angle
CP2 pre-folding cam second angle
RC cam rail
V vertebra
VCF vertebral compression fracture

1. An expandable bone implant (1) for veterinary orthopaedic surgery for restoring the volume and/or geometry of a bone by expansion between a folded configuration and a deployed configuration, said implant comprising a hollow body extending along a longitudinal axis (L) between a proximal end (11) connectable to an implantation instrument (A) for holding the implant (1) and a distal end (12) intended to be first inserted into the bone, wherein:
the wall of said hollow body is formed by a sheet (10) made of a biocompatible metal alloy, closed on itself in a sealed manner, between said proximal (11) and distal (12) ends;
said sheet (10) has, at least in the folded configuration, a plurality of pairs of folds, each of the pairs comprising an antiform fold (101), referred to as convex, and a synform fold (102), referred to as concave, said folds lying on top of each other in the folded configuration so that the surfaces present between each of said convex and concave folds are rolled around the longitudinal axis (L);
said proximal end (11) comprises a ring secured, in a sealed manner, to the lying-down and rolled folds of said sheet (10) over the entire periphery of the proximal end (11), the opening passing through the ring forming an entrance to the inside of the hollow body of the implant (1);
said distal end (12) comprises a cup closing the distal end (12) and secured, in a sealed manner, to the lying-down and rolled folds of said sheet (10) over the entire periphery of the distal end of said hollow body;
said sheet (10) being plastically deformable to allow expansion of the implant from the folded configuration to the deployed configuration, when a fluid is injected into the implant (1).

2. The expandable bone implant according to claim 1, wherein the distance between a synform fold and the next antiform fold is longer than the distance between an antiform fold and the synform fold to make it easier to roll the folds around the longitudinal axis (L).

3. The expandable bone implant according to claim 1, wherein the implant (1) comprises, in the deployed position, a middle portion between its two ends that has the shape of a generalized cylinder, with possible and at least partial persistence of said folds, and at each of its two ends a frustoconical portion connecting the middle portion to the cup and the ring, with a permanent persistence of at least part of the lying-down and rolled folds near the ends.

4. The expandable bone implant according to claim 1, wherein said sheet is plastically deformable also from the folded configuration to the deployed position, in particular as a result of the persistence of the lying-down and rolled folds at the proximal and distal ends, to enable reversibility of the expansion.

5. The expandable bone implant according to claim 1, wherein said cup is adapted to cooperate with the distal end of an implantation instrument (A) passing through said implant via the opening of the proximal end (11), for example by virtue of at least one housing and/or protuberance complementary to at least one protuberance and/or housing of said instrument (A) which comprises a hollow tube (A1) of which the interior canal opens into said hollow body of the implant (1) via at least one opening (A2) allowing said fluid to be injected into the implant (1).

6. The expandable bone implant according to claim 1, wherein said sheet is secured to the ring of the proximal end (11) by a weld (110) that fixes the lying-down and rolled folds against the exterior wall of said ring.

7. The expandable bone implant according to claim 1, wherein said sheet is secured to the cup of the distal end (12) by a weld (120) that fixes the lying-down and rolled folds against the exterior wall of said ring.

8. The expandable bone implant according to claim 1, wherein said sheet is compressed around the ring of the proximal end (11) and/or around the cup of the distal end (12) by a second ring, said a compression ring that holds the lying-down and rolled folds against the exterior wall of said ring and/or of said cup.

9. The expandable bone implant according to claim 1, wherein the folds are, at least in the folded configuration, parallel to the longitudinal axis (L).

10. The expandable bone implant according to claim 1, wherein the outside diameter of said cup and/or of said ring is smaller than or equal to the maximum folded diameter of the implant.

11. The expandable bone implant according to claim 1, wherein the number of pairs of folds is comprised between 3 and 16, generally 4 to 12, and preferably of the order of 8.

12. The expandable bone implant according to claim 1, wherein the sheet (10) is closed on itself by virtue of two folds of opposite directions (synform and antiform) formed on the two opposite edges of the sheet so as to interlock and form a longitudinal closure and impart the shape of a generalized cylinder to the sheet (10), at least prior to folds being produced and rolled.

13. The expandable bone implant according to claim 1, wherein the sheet (10) has a thickness of between 3 and 100 microns, generally between 6 and 50, and preferably 10 and 30 microns.

14. The expandable bone implant according to claim 1, wherein the sheet (10) is made of titanium alloy.

15. The expandable bone implant according to claim 1, wherein the sheet also comprises at least one pair of folds (a synform fold and an antiform fold) of axis not parallel to the longitudinal axis (L), preferably perpendicular so that the implant can expand also lengthwise, or oblique so that the implant can expand in a curved manner.

16. The expandable bone implant according to claim 1, wherein the distance between the folds is variable along the circumference of the implant, so that the shape of the implant in the deployed configuration is curved or asymmetric.

17. The expandable bone implant according to claim 1, wherein said folded diameter is smaller than the deployed diameter by a factor of between 3 and 20, generally 3 to 8, preferably 4 to 7.

18. A system for orthopaedic treatment of damaged bone tissue, comprising a bone substitute cement and at least one instrument (A) for implantation and for injection of cement into the implant, characterized in that it comprises an implant according to claim 1.

19. The system according to claim 18, wherein the instrument for implantation and for injection of cement comprises means for controlling the pressure and/or the aspiration of the cement so that the implant can be re-folded to the folded configuration if necessary.

20. The system according to claim 18, wherein the implantation instrument is different from but complements the injection instrument the cement-injection canal of which passes through a canal inside the rod of the implantation instrument that via its distal end holds the proximal end of the implant.

21. A method for manufacturing an implant according to claim 1, where it comprises:
Closing the sheet on itself and welding it to form a generalized cylinder;
Inserting the closed sheet on a die in the shape of a generalized cylinder having a star-shaped base, the number of branches of the star defining the number of pairs of folds of said sheet of said implant;
Compressing the closed-up sheet between said die and a plurality of projecting elements of a shape that complements the hollows between the branches of the star;
Rolling the folds of said sheet around the longitudinal axis; and
Securing said sheet to the cup and the ring.
